(19) United States
(12) Patent Application Publication
(10) Pub. No.: US 2005/0177883 A1
(43) Pub. Date: Aug. 11, 2005

(54) Systems of transferring embryos and managing recipients

(76) Inventors: Paul C. Rebholtz, Boise, ID (US); Amy K. Rebholtz, Boise, ID (US); George L. Holzer, Boise, ID (US); Kathleen Holzer, Boise, ID (US)

Correspondence Address: KLARQUIST SPARKMAN, LLP, 121 SW SALMON STREET, SUITE 1600, PORTLAND, OR, 97204, US

(21) Appl. No.: 10/470,785

(22) PCT Filed: Jan. 30, 2002
(86) PCT No.: PCT/US02/02930

Related U.S. Application Data
(60) Provisional application No. 60/265,261, filed on Jan. 30, 2001.
(60) Provisional application No. 60/270,453, filed on Feb. 20, 2001.
(60) Provisional application No. 60/312,453, filed on Aug. 14, 2001.

(51) Int. Cl. A01K 67/027

(52) U.S. Cl. 800/15

(57) ABSTRACT

Provided herein are methods of managing embryo transfer programs, for instance bovine embryo transfer programs. Examples of such methods are referred to as “bred recipient transfer” programs, in which embryos (e.g., clonal embryos) are transferred to recipients that have been impregnated. Also provided are methods of enhancing survival of clonal embryos, in which the clonal embryos are transferred to bred recipients, and methods of increasing delivery success rates for cloned animals. Methods of serial transfer of embryos to a recipient, when that recipient returns to heat after an unsuccessful transfer, are provided. Finally, specific methods are provided for cloning male mammals, using diploid differentiated cells taken from a semen sample.

### STATEMENT OF GOVERNMENT SUPPORT

Research and/or development of certain aspects of this invention were funded, at least in part, through Federal SBIR grant number IR43HD38140-01AI, granted through the National Institutes of Health. The government may have certain rights in this invention.

### FIELD

The present disclosure relates to methods of managing embryo recipients in systems of embryo transfer, examples of which methods increase the efficiency of producing animals from transferred embryos, reduce the cost of such programs, and/or increase the birth success rate of such programs. Particular aspects of the disclosure relate to systems of generating and transferring cloned embryos, and managing the recipients thereof.

### BACKGROUND

The birth of the sheep Dolly in 1996 opened the possibility that adult cells could be reprogrammed to act like fertilized embryos and progress, when transferred to a recipient, to the birth of an exact copy of the adult (Wilmut et al., ^{Nature} 385:810-813, 1997). Ashworth et al. (Nature, 394:329-331, 1998) confirmed the authenticity of Dolly's parentage. For some time after the original report on Dolly, numerous laboratories were unable to repeat the experiment. However, the situation changed. Cibelli et al. (Science 280: 1256-1258, 1998) reported the birth of several calves that resulted from the cloning of fetal fibroblast cells. Those fibroblasts carried a “marker” transgene, which conferred resistance to neomycin. The eventual and stated goal of that research is the production of transgenic animals.

Kato et al. (Science 282: 2095-2098, 1998) produced eight calves by cloning tumulus cells and oviduct cells. Wells et al. (Biol. Reprod. 57: 385-393, 1997) produced lambs through cloning of an established cell line using in vivo- and in vitro-produced cytoplasts. A live calf has been cloned from cumulus cells of a 13-year old cow (Wells et al., Biol. Reprod. 60: 996-1005, 1999).

Vignon et al. (Comptes Rendus de I Academie des Sciences Serie III—Sciences de la Vie-Life Sciences. 321: 735-745, 1998) reported two calves produced by nuclear transfer using muscle cells as genetic donors. This group also reported four bovine pregnancies in late gestation. Of these, one originated from a juvenile female skin-cell line and another originated from transgenic fetal skin cells. Zakhartchenko et al. (Mol. Reprod. Dev. 54: 264-272, 1999) produced only a single calf from an adult mammary gland cell and one calf from an adult skin fibroblast. Using goats, Baguisi et al. (Nat. Biotech. 17: 456-461, 1999) produced three kids from fetal somatic cells removed from a transgenic 40-day fetus, which was the product of a mating between a normal female goat and a transgenic male goat. In one of the experimental groups, the couplet was made with an enucleated telophase II oocyte and simultaneously reactivated to induce genome reactivation. Zakhartchenko et al. (J. Reprod. Fertil. 115: 325-331, 1999) also cloned fetal bovine fibroblasts, and then recloned using cells from the resulting morulae. The proportion of couplets developing to blastocysts was significantly improved by the recloning procedure.

Another group has reported that a calf had been born from the cloning of skin fibroblast cells (Yang, Transgenic Animal Res. Conf., Tahoe City, Calif., Aug. 14-19, 1999, oral presentation).

In most published reports, the actual conception rate was low and the number of recipients was very low. However, Wells, et al. (Biol. Reprod. 60: 996-1005, 1999) transferred 100 cloned bovine granulosa cells to recipients. Quiescent cultured adult granulosa cells were fused with metaphase II cytoplasts using a “fusion before activation” procedure. The rate of blastocyst formation was 27.5% (+/−2.5%), similar to that reported previously (Zakhartchenko et al., Mol. Reprod. Dev. 54: 264-272, 1999). After transfer, the 100 recipients produced an initial pregnancy rate of 45%, but only ten calves were born (Wells et al., Biol. Reprod. 60: 996-1005, 1999). This is the largest study reported and confirms the consistent calving rate of approximately 10%.

Cloning of adult cells in cattle has been plagued by low conception rates, high fetal loss rates, and marginal calf survival. From conventional embryo transfer, to frozen embryo transfer, to in vitro produced embryos, to embryos cloned from embryonic cells, and finally embryos cloned from adult somatic cells, conception rate drops, and fetal loss and neonatal calf loss rises. Fetal loss in reported cloning work is often associated with an abnormal allantois and abnormally formed placentomes. These defects suggest that there is inadequate coordination between fetus and mother, rather than a fundamental defect in the cloned fetal tissue.

These major problems with adult cell nuclear transfer (NT) in cattle result in very few of the established pregnancies being maintained beyond sixty days of gestation. In spite of much research, clonal calving rates remain around ten percent. Fetal loss rates and neonatal loss rates are still quite high using the one-step approach. These issues make mass-production of clonal cattle prohibitively expensive; the situation is similar for other animals, livestock and otherwise.

Quite aside from the expenses involved in generating clonal animals, the major expense portion of any embryo transfer (ET) program is the cost and maintenance of recipient animals.

The embryo transfer industry recognizes that transferring twin embryos to a recipient improves the implantation rate for the recipient, and results in the production of some twins. Transferring twin embryos incrementally decreases the number of recipients, and their associated feed and management costs, needed to produce a given number of offspring. Simultaneously, the ratio of the number of recipients to the number of calves is reduced even further. The combined effect is a substantial increase in the efficiency of the ET program coupled with a reduction in the per offspring cost of the program.

Why, therefore, are single embryo transfers the norm in bovine embryo transfer? In the bovine, if the two twins are of different sex, the female will be sterile in almost all cases. If half of the twins are of mixed sex, then approximately half of the females will be sterile. This phenomenon is well known and the females born twin to a bull are called “Freemartins.”

It is also well known in the ET industry that cloned embryos are much less viable than conventional embryos produced by super-ovulation and embryo transfer. Therefore, it is common practice to transfer twin, cloned embryos to each recipient when producing cloned calves. This procedure is successful since, when cloning cattle, the sex of the donor tissue (from which the donor cells are extracted) is known. In instances where the sex is not known, for example when using embryonic cells for the donor material, the cells within a single transfer are at least known to be of the same sex. Consequently, when transferring cloned embryos, both embryos are of the same sex and the Freemartin heifer problem does not develop.

### SUMMARY

The inventors have developed systems for increasing the efficiency of mammalian embryo transfer systems, in some embodiments by increasing the survival rate of embryos, in other embodiments by reducing the costs associated with management of the animals in the system. The methods and systems disclosed herein may be applied to the management of recipient animals of any mammal for the transfer of embryos.

The present disclosure is particularly directed to methods of managing embryo transfer programs, which methods involve breeding (either naturally or with assisted means) a plurality of transfer recipients, thereby generating at least one bred recipient who is implanted with a first embryo. A bred recipient is then identified from among the group, and a second embryo is transferred (ipsilateral or contralateral the first embryo) to the bred recipient, to produce an implanted bred recipient (which may be carrying two embryos). The implanted bred recipient is then monitored throughout her pregnancy to parturition of the resultant one or two offspring. Optionally, the parentage of the offspring is then determined. In specific examples of such methods, one or more recipients that were bred, but which did not implant a first embryo, are recycled into the system for use immediately or later in another breeding and implanting cycle.

Also provided are specific methods of managing a bovine embryo transfer program, which methods involve breeding (either naturally or with assisted means) a plurality of transfer recipient cows, thereby generating at least one bred recipient cow having a first implanted embryo, and identifying at least one bred recipient cow from the plurality within about 14 or fewer days of breeding (e.g., by ultrasound or other means). When a recipient is found to have successfully implanted a first embryo, a second embryo is transferred to that recipient about 6-11 days after heat/estrus to produce an implanted and bred recipient cow, which may be carrying two implanted embryos. These implanted and bred recipients are then evaluated regarding whether the implanted and bred recipient cow remains pregnant, thereby identifying one or more successfully impregnated recipients; the pregnant recipients are then monitored until delivery of at least one offspring. In some examples of such methods, it is further determined which if any of the offspring arose from the transferred bovine embryo.

Methods of enhancing survival of clonal embryos in a mammalian cloning program are also provided. Such methods involve identifying a plurality of bred mammalian recipients, each of which has been impregnated with a first embryo; and transferring a single clonal embryo to each of a plurality of the bred mammalian recipients, thereby enhancing the survival of the clonal embryos. In specific embodiments, the sex of first and/or second embryos in such methods is known prior to implantation.

Also provided are methods of increasing delivery success rates of cloned mammals, which methods involve implanting a sexed in vitro fertilized embryo into a female mammal previously impregnated with a clonal embryo of the same sex as the in vitro fertilized embryo; and monitoring the pregnancy of the female mammal through parturition.

Aspects of the disclosure are also directed to efficient systems for bovine embryo transfer, which systems involve transferring an embryo to a recipient about 21 days after the recipient returns to heat after a prior embryo transfer to that recipient. Such systems can be adapted to other species (other than bovine) by adapting the repeat time for serial transfer of embryos to the recipient, dependent on the length of estrus for that species.

The foregoing and other features and advantages will become more apparent from the following detailed description of several embodiments, which proceeds with reference to the accompanying figures.

### BRIEF DESCRIPTION OF THE FIGURES

FIG. 1 is a flow chart depicting the operation of an embodiment of the bred-recipient embryo transfer management system of the disclosure.

FIG. 2 is a flow chart depicting the operation of an embodiment of the serial embryo transfer system of the disclosure.

### DETAILED DESCRIPTION

I. Abbreviations

DNA deoxyribonucleic acid
ET embryo transfer
GFP green fluorescent protein
IVF in vitro fertilization
PCR polymerase chain reaction
RNA ribonucleic acid
siRNA small inhibitory RNA
   Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the embodiments, the following explanations of terms are provided. These explanations are not intended to limit the listed terms to a scope narrower than would be known to a person of ordinary skill in the fields of animal (e.g., livestock) management, embryo management, embryo transfer, and cloning.

Animal: Living multi-cellular vertebrate organisms, a category that includes, for example, mammals, reptiles, and birds. Animals can also be divided by type, for instance livestock animals (such as cattle, pigs, horses, goats, sheep, fowl, etc.), laboratory test animals (such as mice, rats, and monkeys), domestic animals (such as household cats and dogs) and captive wild animals (such as may be found in a zoological park). Another category of animals is the ruminants, which are animals that chew their own cud (regurgitate and re-chew previously swallowed food). Goats, sheep, cattle, camels, llamas, elk, deer, and antelope are ruminants.

The phrase “group of animals” refers to any set of two or more animals. A group of animals can be, for instance, as few as two animals or as many as hundreds of thousands. Within any group of animals, all of the animals in a group can be of multiple species (cattle and sheep) or more commonly one species (e.g., all cattle). Additionally, a group can include different varieties or breeds of a single species.

In some embodiments, at least one individual animal within a group is identifiable in some reliable way, such that data taken regarding the animal 's characteristics can be correlated with that particular animal. More than one animal within the group, and in some instances all animals of the group, will be individually labeled so they can be correlated with measured data, such as measurements of pre- or post-mortem characteristics. Labeling devices can be anything that will reliably permit correlation, and can include tags attached to the animals (either directly to the animal by way of a piercing, or otherwise such as tied on), brands or dye-stamps (e.g., numbered brands or stamps), implants (for instance implants that include a microchip that is programmable with identifying information), electronic identification tags, etc.

Bred recipient: A female mammal intended to be used in an embryo transfer program which animal has been bred prior to a subsequent transfer of an embryo into the female's uterus. The term “bred” or “breeding” as used here is intended to be broad, and includes natural breeding of the recipients as well as assistive reproductive breeding techniques (e.g. in vitro fertilization and implantation of the resultant embryo(s), artificial insemination, and implantation with clonal embryo(s)).

Cattle: General term used to refer to bovine animals, of the genus Bos. Most domesticated cattle are members of the species Bos taurus and B. indicus. A grown male is referred to as a bull; a grown female, a cow; an infant (of either gender), a calf; a female that has not yet given birth, a heifer; and a young, castrated male, a steer. A bullock is a bull in which the testicles have been pushed up against the body of the animal and the scrotum removed, to maintain the testicles at a higher temperature, thereby reducing the violent behavior tendencies of the animal. The term cattle, as used herein, generally refers to all varieties of cattle, as well as crossbred cattle (hybrids between two varieties or two species) and bovine animals of undetermined heritage.

Clone/cloned/cloning: Cloning is the creation of a living animal/organism that is genetically essentially identical to the unit or individual from which it was produced. The process of two-step cloning may be used with certain methods, using, for instance adult cells, or adult cells in the first round of cloning followed by fetal cells in the second round of cloning. Other cloning techniques, including simple nuclear transfer, may also be used. In many cloning methods, the clone is not precisely genetically identical to the source organism, for instance due to one or more cytoplasmic genetic elements (e.g., mitochondrial genetic elements) introduced with the recipient cytoplasm. Techniques for mammalian cloning are known, and details can be found for instance in the following patent publications:
U.S. Pat. No. 5,945,577: CLONING USING DONOR NUCLEI FROM PROLIFERATING SOMATIC CELLS;
U.S. Pat. No. 6,011,197: METHOD OF CLONING BOVINES USING REPROGRAMMED NON-EMBRYONIC BOVINE CELLS;
U.S. Pat. No. 6,013,857: TRANSGENIC BOVINES AND MILK FROM TRANSGENIC BOVINES;
U.S. Pat. No. 6,147,276: QUIESCENT CELL POPULATIONS FOR NUCLEAR TRANSFER IN THE PRODUCTION OF NON-HUMAN MAMMALS AND NON-HUMAN MAMMALIAN EMBRYOS;
U.S. Pat. No. 6,215,041: CLONING USING DONOR NUCLEI FROM A NON-QUIESCENT SOMATIC CELLS;
U.S. Pat. No. 6,235,969: CLONING PIGS USING DONOR NUCLEI FROM NON-QUIESCENT DIFFERENTIATED CELLS;
U.S. Pat. No. 6,252,133: UNACTIVATED OOCYTES AS CYTOPLAST RECIPIENTS OF QUIESCENT AND NON-QUIESCENT CELL NUCLEI, WHILE MAINTAINING CORRECT PLOIDY;
U.S. Pat. No. 6,258,998: METHOD OF CLONING PORCINE ANIMALS;
U.S. Pat. No. 6,331,659: CUMULUS CELLS AS NUCLEAR DONORS;
U.S. App. No. 20010044937: METHODS FOR PRODUCING TRANSGENIC ANIMALS;
WO 97/07669: QUIESCENT CELL POPULATIONS FOR NUCLEAR TRANSFER;
WO 98/39416: METHOD OF CLONING ANIMALS;
WO 99/34669: CLONING USING DONOR NUCLEI FROM DIFFERENTIATED FETAL AND ADULT CELLS;
WO 00/18902: METHOD OF SCREENING FOR LARGE OFFSPRING SYNDROME; and
WO 01/73107: PRION-FREE TRANSGENIC UNGULATES.
   Depending on the technique, quiescent or proliferating cells can be used in the cloning process. In certain methods, it is beneficial to arrest a proliferating cell (for instance by nutrient deficit or chemical or drug treatment, such as treatment with cytochalasin) during the cloning process.

Contralateral: Literally, on the other side of the body. In discussing embryo transfer into an animal that has a bifurcated uterus (e.g. a cow or a cat), contralateral refers to transferring an embryo into the uterine horn on the opposite side of the body as something. For example, a second embryo could be transferred (and implant) contralateral (on the opposite side from) a first embryo.

Couplet: A fused cell, produced through laboratory-assisted means (e.g., nuclear transfer followed by cell fusion, etc.), that contains cytoplasm that is not native to the nucleus. This can be accomplished by transferring the nucleus, or nuclear material, of one cell, or an entire cell, into another (usually enucleated) cell, such as an enucleated oocyte.

Ipsilateral: Literally, on the same side of the body. In discussing embryo transfer into an animal that has a bifurcated uterus (e.g., a cow or a cat), ipsilateral refers to transferring an embryo into the uterine horn on the same side of the body as something. For example, a second embryo could be transferred (and implant) ipsilateral (on the same side as) a first embryo.

Nuclear transfer: For the purposes of this discussion, nuclear transfer (NT) means fusion of nuclear material (e.g., an isolated nucleus or an entire cell) of a donor cell with an enucleated oocyte so that it is reprogrammed to function like a fertilized embryo. This technique is now known, and details can be found for instance in the following publications: Stice et al., Theriogenology 49:129-138, 1998; Solter, Nature 394:315-316, 1998; Wakayama et al., Nature 394, 369-374, 1998; Wells et al., Biol. Reprod. 57:385-393, 1997; Wilmut et al., Nature 385:810-813, 1997. In particular, nuclear transfer has been used, with moderate success, to produce clonal cattle (see, Lanza et al., Science 288:665-669, 2000; Wells et al., Biol. Reprod. 60:996-1005, 1999; Kato et al., Science 282:2095-2098, 1998; Zakhartchenko et al., Mol. Reprod. Del. 54:264-272, 1999; Zakhartchenko et al., J. Reprod. Fert. 115:325-331, 1999; Kato et al., Science, 282:2095-2098, 1998; Lanza et al., Science, 288:665-669.

Parturition: The act or process of giving birth to one or more offspring.

Preserving: The general term preserving, or preservation, as used herein, refers to scientifically acceptable methods for maintaining a biological sample (such as a cell sample, or a sample of an in vitro cell culture) for an extended period of time, such that the sample (or a cell within the sample) is viable at the end of the period. The period of time will vary with the purpose for which the sample is preserved, and the manner of preservation, and may vary from a few hours to weeks or even months. Methods of preserving biological samples, such as cell and tissue samples and in vitro cultures, are various, and include immortalization of cell cultures, cryopreservation (freezing), and/or lyophilization (freeze-drying).

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Comprising means including. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. In case of conflict, the present specification, including explanations of terms, will control. In addition, the materials, methods, and examples are illustrative only and not intended to be limiting.

III. Overview of Several Embodiments

Providing herein in a first embodiment is a method involving transferring at least one embryo (e.g., a bovine embryo, though it can be any mammal) to at least one bred recipient female, which female already has been implanted with a first implanted, to produce an implanted and bred recipient female animal. The implanted and bred recipient(s) are then evaluated to determine whether they maintain at least one implanted embryo (and thus are still impregnated). At least one of the implanted and bred recipients found to maintain at least one implanted embryo is then allowed to reach parturition. Those implanted and bred recipients, or bred recipients before implantation, that do not maintain at least one implanted embryo, can be recycled into the program for subsequent transfer of a subsequent embryo. Examples of this method are methods of managing an embryo transfer program; specific examples are methods of managing a bovine embryo transfer program.

In specific examples of such methods, the method further involves, prior to transferring at least one second bovine embryo, providing a plurality of transfer recipient cows, breeding (either naturally or by assisted means, such as artificial insemination, impregnation with an in vitro fertilized embryo, or impregnation with a clonal embryo) the plurality of transfer recipient cows, thereby generating the at least one bred recipient cow having a first implanted embryo; and identifying at least one bred recipient cow from the plurality (to which the second embryo can then be transferred).

This disclosure also provides methods that involve identifying a plurality of bred mammalian recipients, each of which has been impregnated with a first embryo, and transferring a single clonal embryo to each of a plurality of the bred mammalian recipients. Examples of such methods are methods of enhancing survival of clonal embryos in a mammalian cloning program, in particular a bovine cloning program.

In particular examples of these methods, the bred recipients have been impregnated by natural breeding. In other examples, the bred recipients have been impregnated using artificial insemination or implantation of an in vitro fertilized embryo or a clonal embryo.

This disclosure further provides methods of managing a bovine embryo transfer program, involving breeding a plurality of transfer recipient cows, thereby generating at least one bred recipient cow having a first implanted embryo (having been bred either by natural or assisted means); identifying at least one bred recipient cow from the plurality within about 14 or fewer days of breeding (e.g., using ultrasound detection of a fetus); transferring a second bovine embryo to the at least one bred recipient cow about 6-11 days after heat/estrus to produce an implanted and bred recipient cow; and evaluating whether the implanted and bred recipient cow remains pregnant, thereby identifying one or more successfully impregnated recipients. Examples of such methods further involve monitoring the one or more successfully impregnated recipients until delivery of at least one offspring; and determining which if any of the offspring arose from the transferred bovine embryo.

A further provided embodiment is a system for bovine embryo transfer, comprising transferring an embryo to a recipient about 21 days after the recipient returns to heat after a prior embryo transfer to that recipient. Specific examples of this system involve transferring a first embryo to a bovine recipient at day about seven counted from estrus, determining if the bovine recipient returns to estrus at about day 21, and transferring a second embryo to the bovine recipient at about day 28, if the bovine recipient was determined to return to estrus at about day 21. The system further involves determining of the bovine recipient returns to estrus at about day 42, and transferring a third embryo to the bovine recipient at about day 49, if the bovine recipient was determined to return to estrus at about day 32. Optionally, the first second, and/or third embryo is a clonal embryo (thus, any one, any two, or all three of the embryos are clonal in different examples).

Specific examples of this system further involve, implanting a second embryo into the recipient. This second embryo in some examples is a clonal embryo.

The disclosure further provides a method, which method involves implanting a sexed in vitro fertilized embryo into a female mammal previously impregnated with a clonal embryo of the same sex as the in vitro fertilized embryo; and allowing the female mammal to proceed to parturition, thereby producing at least one offspring. Examples of this method are methods of increasing delivery success rate of cloned mammals.

A further provided embodiment is a method of producing a clone of a male mammal, which involves isolating from a semen sample (fresh, frozen, or archival, for example) a diploid differentiated cell having a nucleus, and using the nucleus from the isolated diploid differentiated cell in a cloning procedure (for instance, nuclear transfer), thereby producing a clone of the male mammal. Optionally, the male mammal is dead when the clone is produced. In examples of such methods, the male mammal is a bull, a pig, a horse, a goat, a sheep, a mouse, a rat, a monkey, a cat, or a dog. In other examples, the male mammal is a captive wild animal or an endangered species. In one specific example of this method, the male mammal is a bull.

In specific examples of the provided method of producing a clone of a male animal, the differentiated diploid cell is a somatic seminal vesicle cell, a prostate gland cell, a Cowper's gland cell, a Sertoli cell, a white blood cell, an epididymus cell, a urethra cell, or a bladder cell.

Embryos (clonal or non-clonal) for use in the provided methods and systems can be fresh or frozen, transgenic or not transgenic. In those embodiments where the embryo is transgenic, they can be transgenic for a marker, a functional gene, or both.

Transfer of the second embryo to the recipient in the provided methods will be ipsilateral or contralateral the first implanted embryo.

In specific examples of the provided methods, the sex (gender) of the embryos is known prior to their being implanted. Embryo sex can be determined using known means. In specific examples, the embryo (for instance in some embodiments the clonal embryo or the first implanted embryo) is determined to be male prior to transfer. In others, the clonal embryo (for instance in some embodiments the clonal embryo or the first implanted embryo) is determined to be female prior to transfer. In some methods, the sex of both the first and the second embryos is known; in such methods, the sex is the same for some embodiments, and different for others.

Also provided are methods where recipients are bred using artificial insemination, where the artificial insemination comprises insemination with sexed semen. These methods result in embryos of determined sex, since the sex of the semen can be selected.

In the provided embodiments, optionally the parentage of at least one of the resultant offspring is determined, for instance by analyzing or detecting marker genes.

In examples of the provided methods and systems, mammals (for instance, cloned mammals) specifically include cattle, pigs, horses, goats, sheep, mice, rats, monkeys, cats, and dogs. In other examples, mammals (for instance, cloned mammals) are members of a species of wild animals, members of an endangered species, or members of a species of livestock.

IV. Embryo Transfer and Recipient Management Systems.

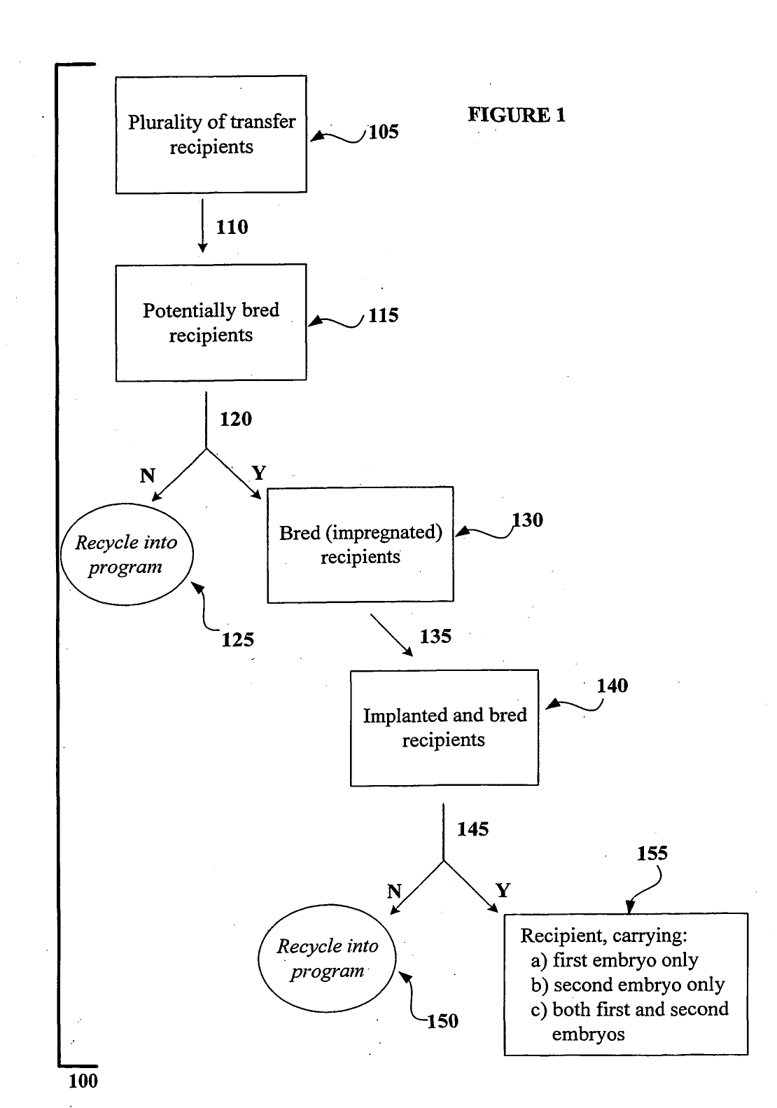

This disclosure provides systems and methods for embryo transfer and recipient management. Though only two specific embodiments are illustrated, additional embodiments are provided. By way of example, one embodiment of a system for embryo transfer and recipient, referred to as bred recipient transfer, is depicted in FIG. 1. In system 100, a plurality of transfer recipients 105 are bred 110 (for instance, naturally or using assisted means), thereby forming a group of one or more potentially bred recipients 115. It is then determined 120 whether one or more of the potentially bred recipients 115 are pregnant (e.g., by detecting return to estrus, detecting the presence of a fetus, etc.). If a potentially bred recipient 115 is determined 120 not to be pregnant, she can optionally be recycled 125 back into the program, for instance by being again subjected to breeding 110.

At least one potentially bred recipient 115 who is determined 120 to be pregnant, now termed a bred (impregnated) recipient 130, is then implanted 135 with a second embryo, thereby producing an implanted and bred recipient 140. It is then determined 145 whether one or more of the implanted and bred recipients 140 is pregnant (using the same or a different means as in determining 120). If an implanted and bred recipient 140 is determined 145 not to be pregnant, she can optionally be recycled 150 back into the program, for instance by being again subjected to implanting 135. Alternatively, she can be dropped from the program.

If an implanted and bred recipient 140 is determined 145 to be pregnant, this animal can now be referred to as a recipient 155. Recipient 115 is pregnant with at least one embryo/fetus, as illustrated in FIG. 1 and discussed more fully below.

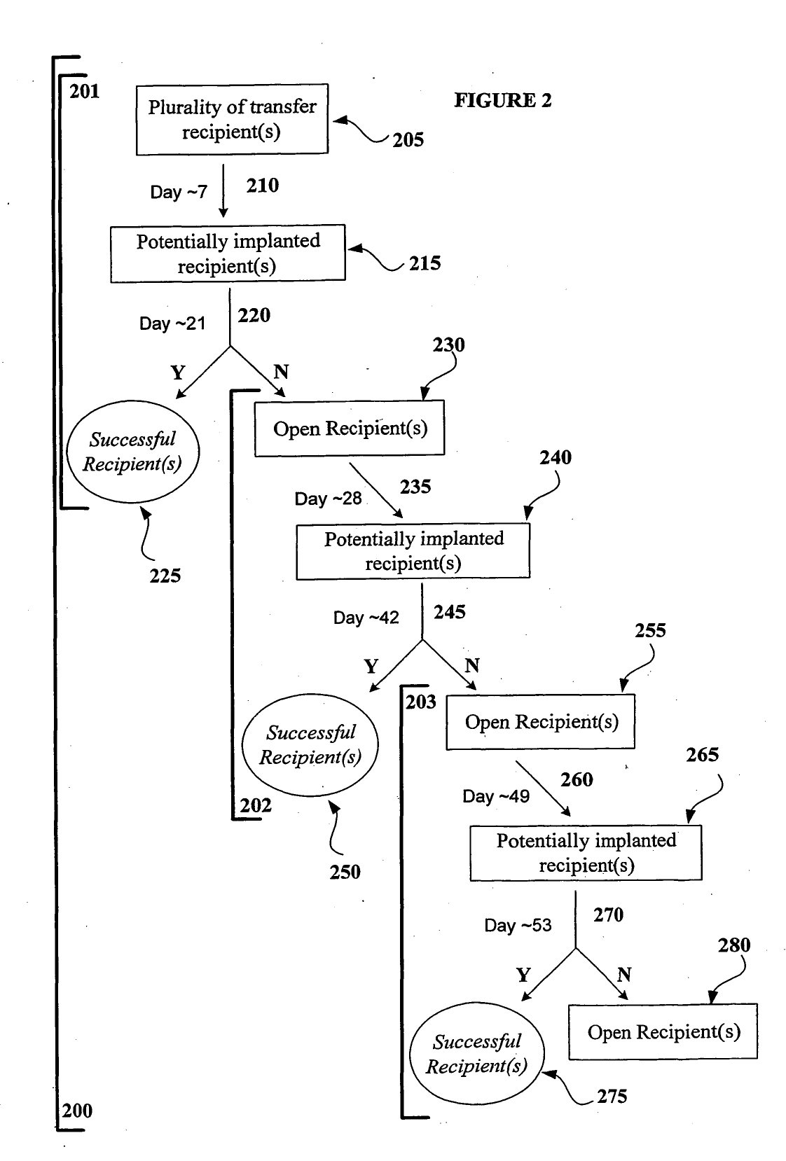

An additional embodiment, referred to as a Triple Transfer management system 200, is illustrated in FIG. 2. As illustrated, system 200 is a bovine Triple Transfer management system; the system can be adapted to other mammalian species by adjusting the timing to fit the estrus cycle in that mammal (not shown). In illustrated system 200, the system begins with a first implantation cycle 201. A plurality of transfer recipients 205 are implanted 210 with a first embryo (for instance, a clonal embryo) at around 7 days, to yield one or more potentially implanted recipient(s) 215. It is then determined 220 whether one or more of the potentially implanted recipient(s) 215 are pregnant (e.g., by detecting return to estrus, detecting the presence of a fetus, etc.). If a potentially implanted recipient 215 is determined 220 to be pregnant, it can be designated a successful recipient 225 and is removed from the cycle.

If, on the other hand, a potentially implanted recipient 215 is determined 220 not to be pregnant, it is termed an open recipient 230 and enters a second implantation cycle 202. A second embryo is implanted 235 into each of one or more open recipients 230, yielding second round potentially implanted recipient(s) 240. Similarly to the first cycle 201, it is then determined 245 whether one or more of the second round potentially implanted recipient(s) 240 are pregnant (e.g., by detecting return to estrus, detecting the presence of a fetus, etc.). If a second round potentially implanted recipient 240 is determined 245 to be pregnant, it can be designated a successful recipient 250 and is removed from the cycle.

If, on the other hand, a second round potentially implanted recipient 240 is determined 245 not to be pregnant, it is termed an open recipient 255 and enters a third implantation cycle 203. A third embryo is implanted 260 into each of one or more open recipients 255, yielding third round potentially implanted recipient(s) 265. Similarly to the first (201) and second (202) cycles, it is then determined 270 whether one or more of the third round potentially implanted recipient(s) 265 are pregnant (e.g., by detecting return to estrus, detecting the presence of a fetus, etc.). If a third round potentially implanted recipient 265 is determined 270 to be pregnant, it can be designated a successful recipient 275 and is removed from the cycle. Otherwise, the third round potentially implanted recipient 265 is an open recipient 280.

Bull Program

This disclosed embodiment comprises a program where cloned male embryos are transferred to recipients that were bred previously, typically about a week previously, when detected in heat. On transfer day (6-11 days after heat/estrus) one cloned bull embryo will be transferred to the uterus of the bred recipient. There are four possible outcomes to this process: (1) Neither embryo survives, and the recipient will return to heat at a date approximately 21 days from the original heat. One embryo survives and the pregnancy is either (2) one natural calf (resulting from the breeding at the original heat) or (3) one cloned calf. (4) Both embryos survive, and the recipient carries twins, one natural calf and one cloned calf together. In this last situation these two calves will both be bulls at a frequency of 50%, and mixed sex at a frequency of 50%. However, the female calves can only be from the natural breeding. These females will be Freemartins and approximately 94% will be sterile. All the bull calves will be of normal fertility regardless of the sex of their co-twin. This method has several clear advantages, including reduced costs (see Table 1).

In examples of this system, all recipients are bred at first heat; no recipients are held unbred for 7 days awaiting a cloned embryo. No recipients are held unbred for 14 more days awaiting return to heat if embryo production does not match recipient availability. All recipients will be put to productive use immediately, thereby increasing the efficiency of the embryo transfer program. Recipients not used for the cloned embryo program have already been bred and can be diverted to a standard breeding program or optionally reused as recipients on the next cycle.

All feed and maintenance costs can be split equitably between the calf raising program and the cloned embryo transfer program. This permits a seamless blending of the cloned embryo transfer program into, for instance, a heifer-raising program. Only recipients pregnant to the original heat date (determined by ultrasound, for instance) need be considered for purchase by a manager of the system. Only the recipients carrying cloned fetuses need be purchased, if the manager is seeking only the clonal offspring. This will result in minimal disruption of a companion commercial calf raising program, and purchase of the cloned pregnancies will represent an additional source of income for the calf raising business.

TABLE 1
TIMELINE FOR BRED RECIPIENT TRANSFER

| Date | Traditional System | | “Bred Recipient Transfer” | |
|---|---|---|---|---|
| 1/1 | Estrus | | Estrus + Breed Recipient - whole herd, | |
| | | | natural heats | |
| 1/8 | Transfer cloned embryo - fraction of | | Transfer cloned embryo - fraction of | |
| | herd* | | herd** | |
| 1/22 | Return to estrus or pregnant | | Return to estrus or pregnant | |
| 2/4 | Pregnancy test with ultrasound | | Pregnancy test with ultrasound | |
| Possible | Pregnant | | pregnant: twins (one natural, one clone) | |
| outcomes: | Open | | pregnant: one clone (temporary, rebred) | |
| | | | pregnant: one natural calf | |
| | | | open (rebred) | |
| 2/7 | Induce estrus on open animals | | | |
| 2/10 | Breed open animals | | | |
| 7/1 | | | Purchase 30 cows with twin pregnancies at | |
| | | | 6 months gestation | |
| | Purchase 20 recipients = | $20,000 | Purchase 30 recipients = | $30,000 |
| | 20 potential pregnancies × 240 d. × $2.50 = | $12,000 | | |
| | 45 days on feed × 100 × $2.50 = | $11,250 | 90 days on feed × 30 × $2.50 = | $6,750 |
| | | | Sell 10 recipients carrying one natural calf = | −$14,000 |
| | SUBTOTAL = | $43,250 | SUBTOTAL = | $22,750 |
| 10/1 | Calve ˜7 recipients with one cloned | | Calve ˜20 recipients with twins | |
| | calf each | | | |
| | | | Sell 20 natural calves of each pair of twins = | −$2,000 |
| | SUBTOTAL = | $43,250 | SUBTOTAL = | $20,750 |
| | COST PER CLONED CALF = | $6,178 | COST PER CLONED CALF = | $1,037 |

This cost increased as impacted by fractional use on date 1/8
*Due to unpredictable supply of embryos, weekends, etc. only a fraction of herd ˜30% will be used at start of program, remainder fed till next cycle.
**Same unpredictability applies, except that unused recipients are bred and additional recipients added as new recipients enter the breeding pen.

By transferring only one cloned embryo to each recipient, fewer cloned embryos are needed per recipient. The natural embryo is effectively used to enhance the survival rate of the cloned embryo to result in a pregnancy. Pregnancy loss appears to be reduced during advanced stages due to the presence of a non-clonal embryo along with a clonal embryo.

Survival of the clonal offspring during delivery is also enhanced, at least in part due to more normal birth weights in the clonal animals, and in part due to beneficial influences on the gestational timing.

TABLE 2
ANALYSIS OF SEX RATIO - CATTLE
NORMAL-NON-IDENTICAL

| Twin #1 | Twin #2 |
|---|---|
| M | M |
| M | F - Sterile |
| F | F |
| F - Sterile | M |

BRED RECIPIENT + CLONED BULL

| Twin #1 | Cloned bull embryo |
|---|---|
| M | M |
| F - Sterile | M |

RECIPIENT BRED FEMALE SEXED SEMEN

| Twin #1 | Cloned female embryo |
|---|---|
| F | F |
| F | F |

| MALES | FEMALES | |
|---|---|---|
| 2 | | |
| 1 | 1 | |
| | 2 | |
| 1 | 1 | |
| 4 | 4 | Note: two females are |
| | | sterile |
| 2 | | |
| 1 | 1 | |
| 3 | 1 | Note: the female is |
| | | sterile |
| | 2 | |
| | 2 | |
| | 4 | Note: there are no sterile |
| | | females |

Heifer Program

This embodiment involves transferring a female cloned embryo. The embryo in place from a natural breeding could be male or female. If the natural calf were male, he would effectively “sterilize” the valuable cloned female fetus. The solution to this problem is to provide sexed female semen for the initial breeding. Sexing semen is in its infancy, but methods of sexing mammal semen are known (see, e.g., U.S. Pat. Nos. 5,135,759 and 6,149,867, herein incorporated by reference in their entirety). Since most markets for sexed semen (particularly in the beef industry) will require male semen, female semen will be a surplus commodity, and therefore more available.

Possible outcomes of this embodiment are as follows: Neither embryo survives, one embryo survives (most commonly this will be the natural female embryo), or both embryos survive, a natural female embryo and cloned embryo. In this scenario, all the fetuses are female and there can be no mixed-sex twins. See Tables 1 and 2.

In addition, embryos of known sex can be used in disclosed embodiments of the recipient management program. For instance, where clonal female animals (e.g., heifers) are desired, a (non-clonal) embryo confirmed to be female could be implanted into an unbred female animal (6-11 days after heat/estrus), along with a cloned female embryo. The non-clonal and clonal embryos can be put into the same or opposite horns of the uterus (where such exists). For instance, the non-clonal embryo can be put in the ipsilateral horn, and the clonal into the contralateral horn, or vice versa, or both can be put into either horn.

The confirmed female embryo could be produced, for instance, through in vitro fertilization (IVF) using sexed semen. Alternatively, the non-clonal fetal embryo could be produced through normal or IVF, followed by sexing of the resultant mixed embryos. Embryos can be sexed using, for instance, PCR analysis or other available technique, such as antibody or karyotyping analysis.

In certain embodiments, the non-clonal embryos (either in vivo or in vitro fertilized) are frozen after samples are taken to determine the gender of the embryo.

Likewise, in a program to produce cloned male animals, confirmed male (or confirmed female) non-clonal embryos can be used.

In all embodiments, genetic markers can be used as an indicator of the parentage of fetuses. One could use a naturally available genetic marker (i.e., Kappa Casein, the Red Factor, and others) to indicate the origin of the fetus present in a single pregnancy. For example, assume that the animal to be cloned is a carrier of the Kappa Casein gene (heterozygous or homozygous). In that situation, one would use as recipients a breed (Holstein) in which the herd is nearly entirely devoid of the Kappa Casein gene. The bull used for natural service would be a bull known not to have the gene. Only the cloned embryo would contain the Kappa Casein gene and an evaluation of fetal tissue would indicate that the fetus was in fact a cloned fetus. This fetal tissue could be fetal cells isolated from the maternal blood or cells removed from an amniocentesis sample. Also, DNA marker analysis in several forms could also be performed on these fetal tissues to determine its match to the donor animal and the donor tissue.

If the animal to be cloned were devoid of the Kappa Casein gene (not heterozygous for the gene), then one would use as recipients a breed of animal that is always positive for the gene, for example Jersey. The bull used to breed the recipients would be homozygous for the gene. If the resulting fetus carried the gene, it would be the natural calf from the breeding. If the fetus did not carry the gene, it would be the cloned calf and could be handled selectively. The same cell analysis from the previous paragraph could be used.

Also provided herein are embodiments of Triple Transfer methods—serial transfer of cloned embryos to recipients every 21 days for those recipients that return to heat on the normal cycle. The usual procedure for managing cattle recipients for an embryo transfer program is to detect heat on the recipients without breeding, and then transferring embryos to them surgically, or non-surgically, approximately seven days later. Then the recipients are tested for the establishment of a pregnancy as soon as practical, more or less 30 days later depending on the technology used. The open recipients may then be recycled and heat detected and reused as recipients. This procedure or a variation of it is used in embryo transfers involving the transfer of fresh embryos, frozen-thawed embryos, in vitro fertilized (IVF) embryos, or cloned embryos. The Triple Transfer procedure embodied in this document is a modification of the recipient management protocol whereby the recipients are reused as surrogates at the first cycle following the original transfer. After the original transfer of an embryo at approximately day 7 (i.e., seven days after the recipient expressed an estrus), the recipients are observed for the next expected estrus. This “return to estrus” will occur in approximately 14 days (21 days from the first estrus). At the appropriate time following the second estrus (i.e., day 7), another embryo can be transferred to the recipient. The concept here is that if the recipient returns to estrus at the next expected estrus period (days 19-22), it is very likely that the first transferred embryo has not survived and the recipient will be eligible for reuse as a surrogate for another embryo. The practical advantage of this process is that the recipient may be reused immediately, without the loss of time involved in a pregnancy test and subsequent recycling of the estrus period. This represents major savings of feed and labor costs on the recipient herd.

Procedures disclosed herein may be applied to the management of recipient animals of any mammal for the transfer of embryos. These embryos may be fresh, frozen, in vitro-fertilized, or cloned. These embryos optionally may be transgenic for marker genes or for functional genes of any source and the associated promoters.

Cloned embryos that are implanted into the uterus of a mammal as described herein may be placed into the same horn of the uterus in which the natural (bred) embryo has implanted (ipsilateral), or into the other horn of the uterus (contralateral). See, for instance, Agca et al. (Theriogenology 50: 129-140, 1998), herein incorporated by reference in its entirety. In specific embodiments, the implanted cloned embryo is planted into the same (ipsilateral) horn.

Each of the methods provided herein also can be employed using in vitro fertilized (IVF) embryos, where the sex of the IVF embryo is known. In some embodiments, the IVF embryos have been fertilized using sexed semen. In other embodiments, the sex of the embryo is determined after fertilization (for instance, using PCR detection of sex-linked genetic markers, or antibody or karyotyping analysis, procedures that are generally carried out about 7 to 8 days after fertilization); such embryos can be created by in vitro or in vivo methods.

In some embodiments, the IVF embryos are also transgenic embryos, for instance embryos which have been rendered transgenic by mixing a desired transgene with the (sexed) semen during in vitro fertilization (semen associated transfection) or injecting the transgene into the ova at or very near IVF using (sexed) bovine semen (transfection by injection). The transgene will be incorporated into the genetic component of the embryo, though integration of the transgene is generally random. An embryo rendered transgenic in this fashion can be employed in any of the methods disclosed herein, for instance with the sexed recipient transfer techniques discussed above.

Without meaning to be limited to one possible explanation, it is currently believed that, since in cattle twins there is usually a sharing of the placental blood supply between twins, the placenta of the normal or IVF twin will assist in the nourishment of the cloned fetus. It is believed that this contributes to better health and survivorship of the cloned fetus.

Also provided are methods that involve using sexed semen to produce normal IVF embryos, then implanting that embryo into the uterus of a mammal (e.g., a cow) into which a cloned embryo of the same sex has already been placed. It is believed that these methods increase the delivery rate of cloned mammals. In one specific example, a known female IVF bovine embryo (made using sexed semen) is placed into the uterus of a cow together with (either contralateral or ipsilateral) a cloned female bovine embryo. Either the IVF or the cloned embryo, or both, is optionally transgenic. These methods permit particularly high efficiency production of cloned animals, as the same sex embryos are both produced in the laboratory; this avoids restrictions or concerns regarding using sexed semen in the field to produce the non-cloned embryo.

V. Cloning Methods

In those embodiments that employ cloned embryos, any method by which an animal can be cloned can be used. Thus, it is contemplated that “cloned embryos” are produced by any conventional method, for instance including the cloning techniques described herein, those described in international patent application PCT/US01/41561, as well as refinements and new cloning techniques. Specifically contemplated herein are methods for cloning a bull, using differentiated diploid cells found in a semen sample.

Cloning of embryos by nuclear transplantation has been developed in several species. Cloning involves the transfer of an adult somatic cell into an enucleated cell, for instance a metaphase II oocyte. This oocyte has the ability to incorporate the transferred nucleus and support development of a new embryo (Prather et al., Biol. Reprod 41:414-418, 1989; Campbell et al., Nature 380:64-66, 1996; Wilmut et al., Nature 385:810-813, 1997). Morphological indications of this re-programming are the dispersion of nucleoli (Szollosi et al., J. Cell Sci. 91:603-613, 1988) and swelling of the transferred nucleus (Czolowska et al., 1984; Stice and Robl, Biol. Reprod 39:657-664, 1988; Prather et al., J. Exp. Zool. 225:355-358, 1990; Collas and Robl. Biol. Reprod 45:455-465, 1991). The most conclusive evidence that the oocyte cytoplasm has the ability to re-program is the birth of offspring from nuclear transplant embryos in several species, including sheep (Smith and Wilmut, Biol. Reprod. 40:1027 1035, 1989; Campbell et al., Nature 380:64-66, 1996; Wells et al., Biol. Reprod. 57:385-393, 1997), cattle (Wells et al., Biol. Reprod. 60:996-1005, 1999; Kato et al., Science 282:2095-2098, 1998; Prather et al., Biol. Reprod. 37:859-866, 1987; Bondioli et al., Theriogenology 33:165-174, 1990), pigs (Prather et al., Biol. Reprod. 41:414-418, 1989) and rabbits (Stice and Robl, Biol. Reprod. 39:657-664, 1988).

Provided herein are methods by which differentiated diploid cells are cultured from a semen sample and used to generate tissue culture, which tissue culture can optionally be used to generate clonal animals. The cultured cells are then cloned, for instance using nuclear transfer to an enucleated, matured oocyte. The cells could be any diploid cells found in the semen sample, including somatic cells from the seminal vesicles, prostate gland, and Cowper's gland, Sertoli cells, white blood cells, and cells sloughed from the epididymus, urethra, and bladder. All of these cells may be present in a sample of semen from, for instance, a bull and could be used as donor cells for the nuclear transfer process. This procedure may be applied to the semen sample, fresh or frozen, of any mammal. By this method, male animals (such as prize or superior animals) can be preserved, even after the animal is deceased, by cloning that male animal from frozen or archival semen samples.

One-Step Cloning

The technique of embryonic cell cloning can be used to reproduce animals selected using the methods described herein. However, to use this cloning technique, the cell sample removed from each animal must be taken from the animal while it itself is embryonic. Thus, in order to use embryonic cloning in the selection and cloning methods of the disclosure, the animals used for the selection must themselves have undergone laboratory manipulation at the embryonic stage, for instance being the result of in vitro fertilization, embryo splitting, or another implantation technique.

In embryonic cell cloning, one or more blastomere cells are removed from a young, e.g. six-day-old, embryo. Using conventional techniques, a blastomere is then immediately fused with an oocyte (unfertilized egg cell), which was harvested from an ovarian follicle and enucleated (the native oocyte nuclear material removed). Using the described selection/cloning system, the blastomere(s) are preserved for a period of time, during which traits of the animal from which the blastomere was removed are examined. Blastomeres that were originally harvested from animals that are later selected using the methods described herein are then taken out of preservation and used for fusion to enucleate oocytes.

After fusion of the blastomere to the enucleate oocyte, the NT embryo is cultured for relatively short time (e.g., five days or so) to determine viability (i.e., development to morula stage). This morula is then implanted into the uterus of a surrogate animal. Clonal animals produced using this technique are exact copies of the original embryo from which the blastomere was removed, except for whatever contribution the enucleate oocyte makes.

In 1997, researchers at the Roslin Institute announced the production of the sheep Dolly by cloning mammary tissue (Wilmut et al., Nature 385:810-813, 1997). Since then several laboratories have reported the production of a fairly small number of calves cloned from adult cells. This process involves the fusing of the nucleus of an adult cell with an oocyte from which all genetic material has been removed (an enucleated oocyte). After short-term in vitro, or in vivo, culture, viable embryos are transferred to surrogate recipient cows for completion of gestation. The initial conception rate for this system has been fairly low and a very large percentage of the pregnancies have been lost before calving.

Cloning can also be performed using the nucleus of an adult cell. In adult cell cloning, an adult somatic cell (i.e. a fibroblast) is fused with an enucleated oocyte. After culture, many of the fused couplets (or cybrids) develop into morulae. When these morulae are transferred to recipient cattle, the reported conception rate is about 30-40%. However, the proportion of the fetuses that persist beyond sixty days gestation has been only about 5-10% (Wells et al., Biol. Reprod. 60:996-1005, 1999).

Two-Step Cloning

Two cycles of cloning can be carried out in order to increase the efficiency of production of cloned calves. This cloning system is referred to herein as “two-step cloning,” “two-cycle cloning,” or “two-step nuclear cloning.”

Two-step cloning involves a first cloning cycle (e.g., by nuclear transfer) using an adult cell, growing the resultant cybrid in vitro and/or in vivo to produce a clonal fetus, then using a fetal cell from the clonal fetus for a second round of cloning (e.g., also by nuclear transfer). This procedure provides more efficient production of calves from adult cells. In one example, a fibroblast from an adult animal is fused with an enucleated oocyte and cultured to about the morula stage. The viable morulae resulting from this procedure are transferred to recipients. Most of these first-cycle pregnancies can be allowed to attempt to reach term, for instance for use as an internal experimental control. After the embryo has developed into a fetus (generally for a sufficient amount of time to display differentiation into tissues and organs), at least one and up to several of these first-cycle fetuses are removed surgically to provide tissue for the production of tissue cultures. By way of example, cattle fetuses can generally be used after they have reached a gestational age of at least 30 days; in specific embodiments, cattle fetuses can be sacrificed at about 45 days gestational age. Any fetal tissue can serve to produce fetal tissue cultures. In representative embodiments, fetal cell cultures are produced from fetal fibroblasts or gonadal cells or cells from the genital ridge. The fetal cell cultures are propagated and samples preserved (e.g., frozen) for future use. In certain embodiments, fetal tissue is used directly for the second round of cloning (without an intervening storage stage, and in some instances without development of an in vitro cell culture).

The fetal cell cultures (e.g., fibroblast cultures) can be used as nuclear donors for the second cloning cycle. In this second cycle (the second “step” of two-step cloning), fetal cultured cells are fused with enucleated oocytes to produce second-generation morulae. These morulae are transferred to recipients and the resulting pregnancies allowed to go to term to produce live progeny. This two-step cloning procedure is expected to result in, for instance, a clonal progeny production rate of 30-40% in cattle, based on conception rates established for embryonic cell cloning. Without meaning to be bound to one theory or explanation, the inventors currently propose that a reprogramming of the genetic clock occurs during early embryonic development and that two cycles of early embryonic development will result in an improved calving rate. The resulting calves are exact copies of the adult animal from which the adult cells were originally removed (except for any influence that may be exerted by cytoplasmic elements introduced during the cloning process).

Adult cells (either proliferating or quiescent) are used as nuclear donors to produce nuclear transfer cloned embryos or fetuses (for instances, fetuses of about 40-45 days). These embryos/fetuses are used to establish cell lines. Cells from the cells lines are then used as nuclear donor cells to produce second generation cloned embryos, which are transferred to recipient animals and carried to term.

As discussed more fully below, the nuclear donor cells in either the first or the second cycle of cloning optionally can be transgenic.

Fibroblasts are proposed as a starting material in certain of the specific embodiments disclosed, since fibroblasts are present in male as well as female specimens. Fibroblasts are readily cultured, but other cell types can be used in the methods described herein.

Pregnancies resulting from the transfer of fetal-origin, second-generation cloned-embryos are allowed to mature for the full gestation period and result in the delivery of live calves.

VI. Transgenesis

Development of transgenic animals that produce therapeutic human proteins provides opportunities to reduce the cost of these products by a factor of ten and in some cases as much as one hundred. Many human genetic diseases exist in which infants are born with a defect in protein metabolism, or a defect in a single protein. Sometimes the cause is genetic, and sometimes there is an error in fetal development. Many hundreds of millions of dollars are spent each year to extract these proteins from blood supplies and cadavers for administration to these patients. Cows or other livestock animals transgenic for these genes can produce many of these proteins in their milk at a fraction of the current cost. Transgenic protein production avoids the risk of disease transmission inherent in products developed from human blood banks and cadavers.

The transfer of DNA into mammalian cells is now a conventional technique. Recombinant expression vectors can be introduced into the recipient cells as pure DNA (transfection) by, for example, precipitation with calcium phosphate (Graham and vander Eb, Virology 52:466, 1973) or strontium phosphate (Brash et al., Mol. Cell Biol. 7:2013, 1987), electroporation (Neumann et al., EMBO J. 1:841, 1982), lipofection (Felgner et al., Proc. Natl. Acad. Sci USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70, 1987). In another embodiment, a cDNA, or fragments thereof, can be introduced by infection with virus vectors. Systems are developed that use, for example, retroviruses (Bernstein et al, Gen. Engr'g 7:235, 1985), adenoviruses (Ahmad et al., J. Virol. 57:267, 1986), or Herpes virus (Spaete et al., Cell 30:295, 1982). Techniques of use in packaging long transcripts can be found in Kochanek et al. (Proc. Natl. Acad. Sci. USA 93:5731-5739, 1996), Parks et al. (Proc. Natl. Acad. Sci. USA 93:13565-13570, 1996) and Parks and Graham (J. Virol. 71:3293-3298, 1997). In yet another embodiment, transgenic sequences can be delivered to target cells in vitro via non-infectious systems, for instance liposomes.

Embodiments described herein thus encompass recombinant vectors that comprise all or part of a desired transgene encoding sequence for expression in a suitable host. The transgene may be operatively linked in the vector to an expression control sequence in the recombinant DNA molecule so that the encoded polypeptide can be expressed. In another embodiment, the expression control sequence may be selected from the group consisting of sequences that control the expression of genes of prokaryotic or eukaryotic cells and their viruses, and combinations thereof. The expression control sequence may be specifically selected from the group consisting of the lac system, the trp system, the tac system, the trc system, major operator and promoter regions of phage lambda, the control region of coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

In particular, the production of transgenic bovines is known. Techniques for producing transgenic bovines can be found for instance in the following: Cibelli et al., Nat. Biotech. 16:642-646, 1998; Cibelli et al., Science 280:1256-1258, 1998; and Brink et al., Theriogenology 53:139-148, 2000.

Transgenics depends heavily on cloning. If embryonic blastomeres are transfected, cloning is used to produce viable embryos. In addition, recent research indicates that transfecting a bed of tissue culture cells with a transgene and a marker gene may increase the efficiency of the transformation process. The development of efficient adult cell cloning procedures will be essential to the implementation of these recent developments. Once a founder transgenic animal is produced, cloning procedures are used to increase the number of animals available, e.g., for the production of therapeutic protein. The selection and cloning methods of the disclosure can be used effectively with transgenic animals and in the production of such animals.

Adult cell cloning applies directly to the field of transgenic production of therapeutic human proteins by cows. The goal is to insert a gene so that a cow will produce a biologically active human protein in her milk. The health aspects of administering transgenically-produced proteins to human patients will require that the producing cattle be specific-pathogen free, to provide assurance that no pathogens are passed with the transgenic protein (e.g., in milk). To ensure this, production of large numbers of cattle embryos by in vitro fertilization of random oocytes collected at slaughterhouses will be replaced with clonal expansion of guaranteed “clean” animals.

In vitro-fertilized one and two cell eggs or embryos are commonly used in the transfection process. The rate of incorporation of the transgene is low using this procedure, and the viability of the embryo is poor. Due to time constraints, expression of the transgene by the embryo cannot be determined before the embryo must be transferred to the recipient. Consequently, embryos expressing the transgene, as well as the large number of embryos not expressing the transgene, must be committed to recipient mothers. The recipient expenses in this situation are therefore huge. The milk producing capability of the resulting transgenic cow is unknown at the outset because she results from the oocyte of an unknown animal randomly selected at the slaughterhouse.

For these and other reasons, the inventors believe that transgenic animals can be produced through transfection of a large number of cultured fibroblast cells removed from the bull or cow with high milk production traits, for instance one selected from a national herd or conglomerate group of animals. The transgene vector may incorporate a marker gene (e.g., for a production of a dye or antibiotic tolerance) that can be used to identify those fibroblasts that have incorporated the transgene. The few cells which effectively incorporate and express the transgene are identified and used as donor cells in the adult cell cloning procedure(s) as described herein, to produce a live calf. This process allows scientists to start with fibroblasts or other cell types from an excellent milk producing animal, screen these cells for any hidden viruses or other pathogens to establish that the cells are specific pathogen free, freeze aliquots of cells, and use them in the transfection process.

Specific lines of these fibroblasts, for instance those that show exceptional cloning capability, can be frozen for repeated use. These fibroblasts will then be transfected with the human or other functional gene and the marker gene. After several days of culture, the transgenic fibroblasts are isolated and cloned. Using this procedure, all of the resulting viable embryos are transgenic embryos. The best of these transgenic embryos can be selected for transfer to recipients.

Using this procedure, the rate of blastocyst formation will not be critical, since all the blastocysts that form are transgenic. The pregnancy rate and fetal survival rate will not need to be comparable to conventional embryo transfer in cattle. However, at this time it is believed that the described two-step cloning procedure will greatly improve the pregnancy rate and fetal survival rate, and possibly the calf survival rate.

Clonal Expansion

As transgenesis progresses, transgenic animals, such as cows and other livestock, can be made to produce virtually any protein. These products would include critical metabolic products, antibacterial agents, antiviral agents, anti-cancer agents, hormones, enzymes and cell growth promoters, and inhibitors. Bacterial, yeast, and even mammalian cell culture systems suffer from the problem of being unable to complete the modification (protein folding and glycosylation) of these products. Often post-translational modifications are essential to maintaining biologic activity in the subject to be treated with the product. By way of example, the bovine mammary gland accomplishes production of complex proteins particularly well, including proper protein folding and glycosylation.

Currently, the production of one transgenic cow successfully incorporates a transgene is a long arduous process requiring thousands of attempts. The cost of producing just one transgenic cow has usually been over four hundred thousand dollars. Once one of these cows is produced, the multiplication of this cow becomes very important.

The commercial application of the described cloning and management techniques for the production and multiplication of transgenic cows provides real and profitable advantages. When transfecting a one-cell or two-cell fertilized embryo, the transgene may be incorporated into only a portion of the embryonic cells (a phenomenon called mosaicism). Mosaicism is very common, and detrimental. Production of a human protein in the milk of a transgenic cow may be low if only 30% of the lacteal cells contain the transgene.

When adult fibroblast cells are transfected and selected for expression of the transgene, each fibroblast gives rise to a cloned calf in whom 100% of the lacteal cells are transgenic. The resulting cow is capable of producing milk much richer in the desired human protein.

Mosaicism creates a similar problem when breeding a transgenic cow to produce transgenic offspring. One would expect that half of the calves of a transgenic cow would contain the transgene. However, if the transgenic cow is a mosaic, then some of her oocytes will contain the transgene and some will not. Due to mosaicism, significantly fewer than half of the calves will be transgenic, since only a portion of the primordial oocytes are actually transgenic.

Cloning also may be essential for the production of herds of cattle from which specific genes have been knocked out (negative or minus transgenics). For example, knocking out the prion gene in cattle would render them immune to bovine spongiform encephalitis (see, e.g. U.S. Pat. No. 5,962,669). Since many human medicines contain products derived from cattle, such as collagen, disease-resistant knockout cattle may be a unique source for certified prion-free medical products (Wilmut, Sci. Am., 279:58-63, 1998).

During transgenesis, the transgene is incorporated into a random chromosome of the very early embryo. If this embryo survives to produce a transgenic cow, the single transgene functions as though it were a dominant gene since there is no matching gene on the homologous chromosome. A transgenic female will produce a hypothetical X milligrams of human protein per milliliter of milk. If one then breeds this cow to an unrelated male (because there exists no other animals with the transgene located at that specific site on that chromosome), both transgenic and non-transgenic calves will result. If one then breeds the transgenic female back to one of her transgenic sons, progeny can be produced that are homozygous for the transgene. This second-generation transgenic animal has two copies of the transgene at the same location on the two homologous chromosomes. Females with two homologous transgenic chromosomes produce 2× milligrams of human protein per milliliter of milk.

Due to the 30-month generation interval in cattle, this procedure is extremely time-consuming. Sperm and egg formation likely will suffer some loss of the transgene. Mosaicism will also decrease the number of eligible matings. Cloning adult tissue cells of original transgenic animals, to produce second generation and homozygous transgenic animals, will be more productive than attempting to increase their numbers by backcross breeding. Though it will still be necessary to backcross the transgenic animal once to achieve homozygosity of the transgene, the cloning techniques described herein can be used to accelerate production of offspring, for instance coupled with in vitro fertilization techniques, once the homozygote is achieved.

Selection of Genes for Transgenesis

Though the description above may be focused on expressing a human protein in a transgenic animal, it is understood that the provided methods are not limited to transgenetic animals expressing human genes. Rather, it is contemplated that diverse transgenes could be used in embryos in the provided methods. In general, transgenes can be viewed as two types of genes—genes that provided function or phenotype to the cell or organism made transgenic (so-called functional genes), and genes that serve as markers for transgenesis. Marker genes include, for instance, a gene coding for a production of a dye or other visually detectable molecule (e.g. a fluorophore such as green fluorescent protein (GFP) or a derivative thereof) or for antibiotic or other drug tolerance or resistance.

Similarly, the term “functional gene” is intended broadly, and is contemplated to include any gene or other nucleic acid sequence that conveys some type of a phenotype on a cell (or animal) into which it is introduced (under appropriate genetic controls). Examples of functional genes include genes that encode structural proteins (e.g., collagen), genes that encode catalytic/enzymatic proteins (e.g., hydrolytic enzymes, biosynthetic enzymes, and so forth), and genes that regulate the expression of other genes. Also encompassed within the term are artificial or engineered (non-native) nucleic acid sequences that encode mutant or variant forms of natural proteins, fragments of natural proteins, and fusions between two or more natural proteins. It is further contemplated that a “functional gene” can be a nucleic acid molecule that reduces the expression of a target gene or protein, for instance an antisense construct, a small inhibitory RNA (siRNA) construct, a dominant negative variant of a protein, and so forth.

This disclosure provides methods for increasing the efficiency and/or reducing the costs associated with an embryo transfer program, particularly a cloning program in mammals such as livestock animals. The disclosure further provides methods for increasing the survival and delivery rates in cloning programs, and methods of cloning male animals from diploid cells found in semen. It will be apparent that the precise details of the methods described may be varied or modified without departing from the spirit of the described invention. We claim all such modifications and variations that fall within the scope and spirit of the claims below.

1. A method of managing a bovine embryo transfer program, comprising:
transferring at least one second bovine embryo to at least one bred recipient cow having a first implanted embryo, to produce an implanted and bred recipient cow;
evaluating whether the implanted and bred recipient cow maintains at least one implanted embryo;
recycling at least one recipient cow that returns to heat into the embryo transfer program; and
allowing the at least one implanted and bred recipient cow to reach parturition.

2. The method of claim 1, further comprising before transferring at least one second bovine embryo:
providing a plurality of transfer recipient cows;
breeding the plurality of transfer recipient cows, thereby generating at least one bred recipient cow having a first implanted embryo; and
identifying at least one bred recipient cow from the plurality.

3. The method of claim 2, where breeding comprises natural breeding.

4. The method of claim 2, where breeding comprises artificial insemination, impregnation with an in vitro fertilized embryo, or impregnation with a clonal embryo.

5. The method of claim 4, where the in vitro fertilized embryo or the clonal embryo is fresh or frozen.

6. The method of claim 4, where the in vitro fertilized embryo or the clonal embryo is transgenic.

7. The method of claim 1, where the second bovine embryo is a clonal embryo.

8. The method of claim 7, where the clonal embryo is transgenic.

9. The method of claim 8, where the clonal embryo is transgenic for a marker gene.

10. The method of claim 8, where the clonal embryo is transgenic for a functional gene.

11. The method of claim 1, where the second embryo is transferred ipsilateral to the first implanted embryo.

12. The method of claim 1, where the second embryo is transferred contralateral to the first implanted embryo.

13. The method of claim 7, where the clonal embryo is determined to be male prior to transfer.

14. The method of claim 7, where the clonal embryo is determined to be female prior to transfer.

15. The method of claim 4, where the artificial insemination comprises insemination with sexed semen.

16. The method of claim 1, where the first implanted embryo is determined to be male prior to implantation.

17. The method of claim 1, where the first implanted embryo is determined to be female prior to implantation.

18. The method of claim 1, further comprising determining parentage of at least one resultant offspring of at least one implanted and bred recipient cow.

19. A method, comprising:
identifying a plurality of bred non-human mammalian recipients, each of which has been impregnated with a first embryo;
transferring a single clonal embryo to each of a plurality of the bred non-human mammalian recipients.

20. The method of claim 19, wherein the method is a method of enhancing survival of clonal embryos in a non-human mammalian cloning program.

21. The method of claim 19, where the bred recipients have been impregnated by natural breeding.

22. The method of claim 19, where the bred recipients have been impregnated using artificial insemination or implantation of an in vitro fertilized embryo.

23. The method of claim 22, where the in vitro fertilized embryo is fresh or frozen.

24. The method of claim 22, where the in vitro fertilized embryo or the clonal embryo is transgenic.

25. The method of claim 19, where the clonal embryo is transgenic.

26. The method of claim 25, where the clonal embryo is transgenic for a marker gene.

27. The method of claim 25, where the clonal embryo is transgenic for a functional gene.

28. The method of claim 19, where the non-human mammalian recipients are bovine.

29. The method of claim 28, where the clonal embryo is transferred ipsilateral to the first embryo.

30. The method of claim 28, where the clonal embryo is transferred contralateral to the first embryo.

31. The method of claim 19, where the clonal embryo is determined to be male prior to transfer.

32. The method of claim 19, where the clonal embryo is determined to be female prior to transfer.

33. The method of claim 22, where artificial insemination comprises insemination with sexed semen.

34. A method of managing a bovine embryo transfer program, comprising:
breeding a plurality of transfer recipient cows, thereby generating at least one bred recipient cow having a first implanted embryo;
identifying at least one bred recipient cow from the plurality within about 14 or fewer days of breeding;
transferring a second bovine embryo to the at least one bred recipient cow about 6-11 days after heat/estrus to produce an implanted and bred recipient cow;
evaluating whether the implanted and bred recipient cow remains pregnant, thereby identifying one or more successfully impregnated recipients;
monitoring the one or more successfully impregnated recipients until delivery of at least one offspring; and
determining which if any of the offspring arose from the transferred bovine embryo;
wherein the second bovine embryo is a clonal embryo.

35. The method of claim 34, where breeding comprises natural breeding.

36. The method of claim 34, where breeding comprises artificial insemination, impregnation with an in vitro fertilized embryo, or impregnation with a clonal embryo.

37. The method of claim 36, where the in vitro fertilized embryo or the clonal embryo is fresh or frozen.

38. The method of claim 36, where the in vitro fertilized embryo or the clonal embryo is transgenic.

39. The method of claim 34, where the clonal embryo is transgenic.

40. The method of claim 34, where the clonal embryo is transgenic for a marker gene.

41. The method of claim 39, where the clonal embryo is transgenic for a functional gene.

42. The method of claim 34, where the second bovine embryo is transferred ipsilateral to the first implanted embryo.

43. The method of claim 34, where the second bovine embryo is transferred contralateral to the first implanted embryo.

44 The method of claim 34, where the clonal embryo is determined to be male prior to transfer.

45. The method of claim 34, where the clonal embryo is determined to be female prior to transfer.

46. The method of claim 36, where the artificial insemination comprises insemination with sexed semen.

47. The method of claim 36, where the first implanted embryo is determined to be male prior to implantation.

48. The method of claim 36, where the first implanted embryo is determined to be female prior to implantation.

49. The method of claim 34, further comprising determining parentage of at least one offspring of at least one implanted and bred recipient cow.

50. The method of claim 34, where identifying at least one bred recipient cow from the plurality within about 14 or fewer days of breeding comprises ultrasound detection of a fetus.

51. A method for bovine embryo transfer, comprising transferring an embryo to a recipient about 21 days after a prior embryo transfer to the recipient, when the recipient returns to heat after the prior embryo transfer to the recipient.

52. The method of claim 51, comprising:
transferring a first embryo to a bovine recipient at day about seven counted from estrus;
determining if the bovine recipient returns to estrus at about day 21;
transferring a second embryo to the bovine recipient at about day 28, if the bovine recipient was determined to return to estrus at about day 21;
determining of the bovine recipient returns to estrus at about day 42; and
transferring a third embryo to the bovine recipient at about day 49, if the bovine recipient was determined to return to estrus at about day 32.

53. The method of claim 52, wherein the first, second, and/or third embryo is a clonal embryo.

54. The method of claim 53, where the first, second, and third embryos are each clonal embryos.

55. The method of claim 52, further comprising:
in at least one bovine recipient that does not return to estrus at 21 or 42 days, implanting a second embryo into the recipient.

56. The method of claim 55, wherein the second embryo is a clonal embryo.

57. A method, comprising:
implanting a sexed in vitro fertilized embryo into a female non-human mammal previously impregnated with a clonal embryo of the same sex as the in vitro fertilized embryo; and
allowing the female non-human mammal to proceed to parturition.

58. The method of claim 57, where the method is a method of increasing delivery success rate of cloned non-human mammals.

59. The method of claim 57, where the sexed in vitro fertilized embryo was generated using sexed semen.

60. The method of claim 58, where the embryos are female.

61. The method of claim 57, where the embryos are male.

62. The method of claim 57, where the cloned mammals are cattle, pigs, horses, goats, sheep, mice, rats, monkeys, cats, or dogs.

63. The method of claim 59, wherein the cloned mammals are cattle.

64. The method of claim 57, where the mammals are members of a species of wild animals, members of an endangered species, or members of a species of livestock.

65. A method of producing a clone of a male non-human mammal, comprising:
isolating from a semen sample from the male non-human mammal a diploid differentiated cell having a nucleus; and
using the nucleus from the isolated diploid differentiated cell in a cloning procedure, thereby producing a clone of the male mammal.

66. The method of claim 65, where the semen sample is a frozen or archival semen sample.

67. The method of claim 65, where the male non-human mammal is dead when the clone is produced.

68. The method of claim 65, where the male mammal is a bull, a pig, a horse, a goat, a sheep, a mouse, a rat, a monkey, a cat, or a dog.

69. The method of claim 65, where the male mammal is a captive wild animal or an endangered species.

70. The method of claim 65, where the male mammal is a bull.

71. The method of claim 65, where the cloning procedure comprises nuclear transfer.

72. The method of claim 65, where the differentiated diploid cell is a somatic seminal vesicle cell, a prostate gland cell, a Cowper's gland cell, a Sertoli cell, a white blood cell, a epididymus cell, a urethra cell, or a bladder cell.
